(19) United States
(12) Patent Application Publication
(10) Pub. No.: US 2012/0021010 A1
(43) Pub. Date: Jan. 26, 2012

(54) ANTIPLATELET AGENT AND METHODS OF USING THE SAME

(75) Inventors: Suryyani Deb, Asansol (IN); Anjan Kr. Dasgupta, Kolkata (IN)

(73) Assignee: University of Calcutta

(21) Appl. No.: 13/056,244

(22) PCT Filed: Jun. 14, 2010
(86) PCT No.: PCT/IB10/01421

§ 371 Date: Jan. 27, 2011

(30) Foreign Application Priority Data
Apr. 29, 2010 (IN) 487/KOL/2010

(51) Int. Cl. A61K 9/14; A61K 31/295; A61K 39/395; A61P 7/02; A61P 9/10; A61P 9/00; A61N 2/00; A61K 33/26; C12Q 1/02; B82Y 5/00

(52) U.S. Cl. 424/400; 424/646; 514/502; 424/133.1; 435/29; 600/9; 977/773; 977/915

(57) ABSTRACT

Disclosed herein are compositions and methods for inhibiting platelet aggregation in a patient in need thereof. The compositions and methods use superparamagnetic iron oxide nanoparticles (SPIONs), which are shown to inhibit aggregation of platelets. Such methods are useful in preventing blood clotting in diseases such as acute coronary syndrome. Also disclosed are in vitro methods of sensing platelet function using SPIONs.

### TECHNICAL FIELD

The present technology relates generally to platelet aggregation inhibitors and methods of using the same. The methods and compositions are advantageously useful for decreasing or preventing platelet aggregation and platelet activation in a patient or a biological sample.

### BACKGROUND

The following description is provided to assist the understanding of the reader. None of the information provided or references cited is admitted to be prior art to the present technology.

Acute vascular diseases, such as acute coronary syndrome (ACS), myocardial infarction, stroke, pulmonary embolism, deep vein thrombosis, peripheral arterial occlusion and other blood system thromboses constitute major health risks. Such diseases are caused by either partial or total occlusion of a blood vessel by a blood clot, which consists of one or both of fibrin and aggregated platelets.

Platelets, or thrombocytes, are small, irregularly-shaped anuclear cells (i.e., cells that do not have a nucleus containing DNA), 2-4 μm in diameter, which are derived from fragmentation of precursor megakaryocytes. The average lifespan of a platelet is between 8 and 12 days. Platelets play a fundamental role in hemostasis and are a natural source of growth factors. They circulate in the blood of mammals and are involved in hemostasis, leading to the formation of blood clots.

Platelets have both a distinct hemostatic function and a thromboplastic function. Hemostasis is initiated within a few seconds following a trauma, when platelets begin to adhere to the edges of the lesion. This initial adherence of platelets may be mediated by collagen exposed at the site of blood vessel wall trauma or by newly generated thrombin. Once in contact with collagen or thrombin, platelets undergo activation, releasing a variety of chemicals, including thromboxane A₂, adenosine diphosphate (ADP), adenosine-5′-triphosphate (ATP), serotonin (5-HT), epinephrine (EPI), and norepinephrine (NE). The released ADP and thromboxane A₂ cause additional platelets from the issuing blood to aggregate to those already attached to the vessel wall. The newly attached platelets also undergo a release reaction and the process continues until a hemostatic platelet plug forms. In addition to releasing ADP and thromboxane A₂, platelets also express platelet factor 3, which promotes the clotting cascade, ultimately resulting in thrombin generation and fibrin deposition at the site of injury. Thrombin also binds to receptors on the platelet membrane and causes further platelet aggregation and release. The ultimate result is a mixed clot composed of aggregated platelets and polymerized fibrin.

EPI and NE can also potentiate platelet aggregation, especially in case of the ACS patients. EPI can reduce aspirin effectiveness in aspirin-treated ACS patients and also can reduce the beneficial effect of clopidogrel in the P2Y12 blockade. Both EPI and NE, acting on alpha2A-adrenoreceptors, have been reported to induce aggregation and to facilitate the aggregation response to ADP, thrombin, and thromboxane.

### SUMMARY

In one aspect, the present disclosure provides a method for inhibiting platelet aggregation in a subject in need thereof, the method comprising: administering to the subject an effective amount of one or more superparamagnetic iron oxide nanoparticles (SPIONs). In one embodiment, the SPIONs at least partially inhibit an epinephrine signaling pathway, thereby inhibiting epinephrine-induced platelet aggregation. In one embodiment, the SPIONs delay epinephrine-induced platelet aggregation. In one embodiment, the subject is an acute coronary syndrome patient.

In one embodiment, the one or more SPIONs are functionalized SPIONs. In one embodiment, the functionalized SPIONs are functionalized with citrate. In one embodiment, the one or more SPIONs have a polydispersity from about 0.25 to about 0.29. In one embodiment, the one or more SPIONs have a mean diameter from about 18 nm to about 22 nm.

In one embodiment, the method further comprises administering to the subject one or more additional anti-platelet aggregation agents. In one embodiment, the one or more additional anti-platelet aggregation agents are selected from the group consisting of: aspirin, clopidogrel, ticlopidine, abciximab, dipyridamole.

A method for treating a thrombotic disorder in a patient in need thereof, the method comprising: administering to the patient an effective amount of one or more superparamagnetic iron oxide nanoparticles (SPIONs).

The method of claim 12, wherein the thrombotic disorder is selected from the group consisting of acute thrombotic stroke, venous thrombosis, myocardial infarction, unstable angina, abrupt closure following angioplasty or stent placement, and thrombosis as a result of peripheral vascular surgery.

In one aspect, the present disclosure provides a method for sensing platelet function, the method comprising: contacting a first sample of platelets from a subject with an agonist of platelet aggregation; contacting a second sample of platelets from the subject with the agonist of platelet aggregation and one or more superparamagnetic iron oxide nanoparticles (SPIONs); measuring the aggregation of the platelets in the first sample and the second sample; and comparing the aggregation of platelets between the first sample and the second sample to determine the response of the sample of platelets to the agonist.

In one embodiment, the agonist is selected from the group consisting of: epinephrine, ADP and collagen. In one embodiment, the subject is an acute coronary syndrome patient that has been administered an antiplatelet aggregation agent. In one embodiment, the anti-platelet aggregation agent is selected from the group consisting of: aspirin, clopidogrel, ticlopidine, abciximab, dipyridamole. In one embodiment, the method further comprises comparing the measured response of the sample of platelets to a reference sample in order to detect an aberrant platelet function.

In one embodiment, the sample is a body fluid sample. In one embodiment, the sample is a whole blood sample or platelet rich plasma. In one embodiment, measuring the aggregation of the platelets is by optical aggregometry.

In one aspect, the present disclosure provides a kit for sensing platelet function, the kit comprising: one or more vials containing superparamagnetic iron oxide nanoparticles (SPIONs); and one or more vials containing one or more agonists of platelet aggregation. In one embodiment, the kit further comprise an optical aggregometer. In one embodiment, the one or more agonists of platelet aggregation are selected from the group consisting of: epinephrine, ADP and collagen.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE FIGURES

FIG. 1 shows an illustrative embodiment of a dynamic light scattering study showing the nanoparticle size.

FIG. 2A shows an illustrative embodiment of platelet aggregation by arachidonic acid (0.5 mM) in the presence and absence of SPIONs. FIG. 2B shows an illustrative embodiment of platelet aggregation by ADP (10 μM) in the presence and absence of SPIONs.

FIG. 3A shows an illustrative embodiment of platelet aggregation by epinephrine (10 μM final concentration) in the presence and absence of SPIONs for a clopidogrel responder patient. FIG. 3B shows an illustrative embodiment of platelet aggregation by epinephrine (10 μM final concentration) in the presence and absence of SPIONs, before clopidogrel treatment. The inset figure shows ADP-induced aggregation in a patient not treated with clopidogrel. FIG. 3C shows an illustrative embodiment of platelet aggregation by epinephrine (10 μM final concentration) in the presence and absence of SPIONs, after clopidogrel treatment. The inset figure shows ADP cannot induce aggregation in a patient treated with clopidogrel.

FIG. 4 is a graph showing the effects of illustrative dextrin functionalized SPIONs on platelet aggregation.

FIG. 5 is a series of graphs showing the effects of illustrative citrate functionalized SPIONs and citrate buffer on platelet aggregation.

### DETAILED DESCRIPTION

In the following detailed description, reference may be made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented here. In the description that follows, a number of terms are used extensively. The terms described below are more fully understood by reference to the specification as a whole. Units, prefixes, and symbols may be denoted in their accepted SI form.

The terms “a” and “an” as used herein mean “one or more” unless the singular is expressly specified. Thus, for example, reference to a “nanoparticle” includes a mixture of two or more such nanoparticles, as well as a single nanoparticle.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the “administration” of an agent to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

As used herein, the term “effective amount” refers to a quantity sufficient to achieve a desired therapeutic and/or prophylactic effect, e.g., an amount which results in the prevention of, or a decrease in, the symptoms associated with a disease, e.g., acute coronary syndrome. The amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the term “nanoparticle” refers to any material having dimensions in the 1-1,000 nm range. In some embodiments, nanoparticles have dimensions in the 2-200 nm range, in the 2-150 nm range, or in the 2-100 nm range. Nanoparticles include, but are not limited to, nanoscale materials such as superparamagnetic nanoparticles. In one embodiment, the nanoparticles are physiologically acceptable.

As used herein, the term “superparamagentic iron oxide nanoparticle” or “SPION” refers to superparamagnetic iron oxide crystalline structures that have the general formula [Fe₂⁺O₃]ₓ[Fe₂⁺O₃(M²⁺O)]₁₋ₓ where 1≧x≧0 M²⁺ may be a divalent metal ion such as iron, manganese, nickel, cobalt, magnesium, copper, or a combination thereof. In one embodiment, when the metal ion (M²⁺) is ferrous ion (Fe²⁺) and x=0, the SPION is magnetite (Fe₃O₄). In general, superparamagnetism occurs when crystal-containing regions of unpaired spins are sufficiently large that they can be regarded as thermodynamically independent, single domain particles called magnetic domains. These magnetic domains display a net magnetic dipole that is larger than the sum of its individual unpaired electrons. In the absence of an applied magnetic field, all the magnetic domains are randomly oriented with no net magnetization. Application of an external magnetic field causes the dipole moments of all magnetic domains to reorient resulting in a net magnetic moment.

As used herein, the term “polydispersity” generally refers to variability of component size within a given sample. The polydispersity of a nanoparticle composition may be shown by transmission electron microscopy (TEM). The polydispersity of the nanoparticles compositor may also be measured using dynamic light scattering to determine the hydrodynamic diameter (D_{H}).

As used herein, the terms “treating” or “treatment” or “alleviation” refers to both therapeutic treatment and prophylactic or preventative measures, wherein the object is to prevent or slow down (lessen) the targeted condition, e.g., platelet aggregation. For example, subject is successfully “treated” for platelet aggregation accompanying ACS if, after receiving a therapeutic amount of the agents according to the methods described herein, the subject shows observable and/or measurable reduction in or absence of one or more signs and symptoms of ACS. It is also to be appreciated that the various modes of treatment or prevention of medical conditions as described are intended to mean “substantial”, which includes total but also less than total treatment or prevention, and wherein some biologically or medically relevant result is achieved.

As used herein, “prevention” or “preventing” of a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “drug,” “compound,” “active agent,” “actives,” “pharmaceutical composition,” “pharmaceutical formulation,” and “pharmacologically active agent” are used interchangeably herein to refer to any chemical compound, complex or composition, charged or uncharged, that is suitable for administration and that has a beneficial biological effect, suitably a therapeutic effect in the treatment of a disease or abnormal physiological condition, although the effect may also be prophylactic in nature. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent,” and “drug” are used, then, or when a particular active agent is specifically identified, it is to be understood that applicants intend to include the active agent per se as well as pharmaceutically acceptable, pharmacologically active salts, esters, amides, prodrugs, conjugates, metabolites, analogs, etc.

As used herein, the term “subject” refers to a mammal, such as a human, but can also be another animal such as a domestic animal (e.g., a dog, cat, or the like), a farm animal (e.g., a cow, a sheep, a pig, a horse, or the like) or a laboratory animal (e.g., a monkey, a rat, a mouse, a rabbit, a guinea pig, or the like). The term “patient” refers to a “subject” who is, or is suspected to be, afflicted with a disease or condition, such as ACS.

The terms “optional” and “optionally” mean that the subsequently described circumstance may or may not occur, so that the description includes instances where the circumstance occurs and instances where it does not.

### Anti-Platelet Compositions and Methods

The present technology relates to the inhibition of platelet aggregation by administration of certain nanoparticle compositions to a subject in need thereof. In some embodiments, the nanoparticle compositions are administered. Also provided is a method for the treatment or prevention of acute coronary syndrome.

In one embodiment, the present disclosure provides a method for inhibiting platelet aggregation in a subject in need thereof, the method comprising: administering to the subject an effective amount of one or more superparamagnetic iron oxide nanoparticles (SPIONs). In some embodiments, the SPION at least partially inhibits an epinephrine signaling pathway, thereby inhibiting epinephrine-induced platelet aggregation. Epinephrine is an important agonist for platelet activation, particularly in acute coronary syndrome (ACS) patients. Epinephrine is substantially increased during stress, exercise or smoking and may result in clinically important platelet activation. Thus, the present methods may cover platelet aggregation associated with many types of acute vascular diseases, such as ACS, myocardial infarction, stroke, pulmonary embolism; deep vein thrombosis, peripheral arterial occlusion and other blood system thromboses constitute major health risks. In some embodiments, the present methods may cover other conditions involving unwanted platelet aggregation. For example, certain medical and surgical procedures and medical conditions may cause unwanted platelet aggregation in individuals. Accordingly, the disclosure features a method for inhibiting platelet aggregation in patients at risk thereof.

In one embodiment, the methods include the intravenous, subcutaneous, or oral administration of one or more SPIONs, e.g. citrate-functionalized SPIONs, to a subject. As described above, the methods may be used in conjunction with a medical or surgical procedure or in treatment for an adverse medical condition.

Any nanoparticle which meets the size and magnetic criteria can be used as a component of the therapeutic compositions disclosed herein. In one embodiment, the nanoparticles are magnetic nanoparticles. In one embodiment, the nanoparticles are superparamagnetic. Super paramagnetic particles are crystalline particles of a magnetic medium that are so small that their magnetization can randomly flip direction under the influence of temperature. Nanoscale superparamagnetic particles are also known as super paramagnetic nanoparticles (SPN). Super paramagnetic nanoparticles may include, for example, iron oxide or nickel ferrite.

Nanoscale iron oxide super paramagnetic particles as also known as super paramagnetic iron oxide nanoparticles (SPIONs). In one embodiment, the SPION may be a colloidal SPION. A colloidal SPION is a mixture of SPIONs in a continuous liquid phase. In order to maintain the continuous liquid phase, the nanoparticle may have a surface potential. For example, if the surface potential is on the order of 20 mV, the electric repulsion is strong enough to maintain the colloidal state. In SPIONs, apart from the hydrophobic interaction between different colloidal particles, the magnetic interaction between them also plays a role in maintaining the colloidal stability. A surface potential can originate from surface functionalization.

In some embodiments, SPIONs may have different surface functionalization. Generally bare nanoparticles are toxic to cells, so the surface of nanoparticles may be functionalized with agents which are nontoxic and biocompatible. Example agents include, but are not limited to, citrate, polyethylene glycol (PEG), polyvinyl alcohol (PVA), dextran. In one embodiment, the SPIONs are functionalized by citrate. Additional examples of surface functionalization are shown in Table 1.

TABLE 1
Illustrative classes of surface functionalization for SPIONs

| | | |
|---|---|---|
| Natural Polymers | Starch | Good for MRI and drug delivery |
| | Gelatin | Used as a biocompatible gelling agent |
| | Chtitosan | Non-Viral gene delivery system |
| Synthetic Polymer | PEG | Improves blood circulation |
| | PVA | Prevents agglomeration |
| | PLA | Improves biodegradability |
| | Alginate | Improves Stability |
| | PMMA | Used as thermo sensitive drug delivery |
| | | system |
| | PAA | Improves Stability as well as |
| | | bio-conjugation |

The SPIONs for use in the present methods can be prepared and stored by any method known to those of skill in the art. Commercially prepared SPIONs can also be used. An illustrative method for preparing SPIONs is described in Example 1.

The SPIONs described herein may be administered as a monotherapy or in combination. Thus, the methods disclosed herein may employ a variety of therapeutic agents in combination with the one or more SPIONs. Illustrative platelet activation or aggregation inhibitors include glycoprotein IIb/IIIa antagonists, heparins, tissue plasminogen activator, Factor Xa inhibitors, purinergic-receptor antagonists, thrombin inhibitors, phosphodiesterase inhibitors (e.g., dipyridamole), cyclooxygenase inhibitors (e.g., aspirin), CD40 antagonists, and leukotriene inhibitors. In addition, platelet activation or aggregation inhibitors may be administered with other compounds, such as those that lower cholesterol, e.g., statins (such as, atorvastatin, fluvastatin, lovastatin, pravastatin, cerivastatin, rosuvastatin, and simvastatin), nicotinic acid drugs (such as, Advicor, Niacin, and Niaspin), drugs that sequester bile acid (such as, colestipol, cholestyramine, and colesevelam), and fibrates (such as, clofibrate, gemfibrozil, and fenofiribrate).

Two or more agents may be administered concomitantly in the same dose or in separate doses. Agents in combination may also be administered at different times as appropriate. In one embodiment, SPIONS, e.g., citrate-functionalized SPIONs, are co-administered with aspirin and a heparin, e.g., a low molecular weight heparin.

### Pharmaceutical Formulations

Pharmaceutical compositions may include an effective amount of one or more nanoparticles or additional agent(s) dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains one or more nanoparticles or additional agent(s) will be known to those of skill in the art in light of the present disclosure, as exemplified by Remington's Pharmaceutical Sciences, 18th Ed. Mack Printing Company, 1990. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The pharmaceutical composition may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art.

The actual dosage amount of a nanoparticle composition administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of the nanoparticles. In other embodiments, the nanoparticles may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g. triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. One may also include isotonic agents, such as, for example, sugars, sodium chloride or combinations thereof.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the typical methods of preparation are vacuum-drying or freeze-drying techniques which yield a powder of the active ingredient plus any additional desired ingredient from a previously sterile-filtered liquid medium thereof. The liquid medium should be buffered if necessary and the liquid diluent first rendered isotonic prior to injection with sufficient saline or glucose.

### Sensing Methods

In one aspect, SPIONs may be used in diagnostic methods for monitoring epinephrine-induced aggregation of platelets. In one embodiment, the method is an in vitro assay that involves contacting a first sample of platelets from a subject with an agonist of platelet aggregation; contacting a second sample of platelets from the subject with the agonist of platelet aggregation and one or more superparamagnetic iron oxide nanoparticles (SPIONs); measuring the aggregation of the platelets in the first sample and the second sample; and comparing the aggregation of platelets between the first sample and the second sample to determine the response of the sample of platelets to the agonist.

Aggregation of platelets can be measured by various procedures. In vitro platelet aggregation is a laboratory method used to assess the in vivo ability of platelets to form the aggregates leading to a primary hemostatic plug. In this technique, an aggregating agent such as epinephrine is added to whole blood or platelet rich plasma (PRP) and aggregation of platelets monitored. Platelet aggregometry is a diagnostic tool that can provide insights difficult to obtain by other techniques, thus aiding in patient diagnosis and selection of therapy. Currently there are two detection methods used in instruments with FDA clearance for performing platelet aggregometry: optical and impedance measurements. Optical detection of platelet aggregation is based on the observation that, as platelets aggregate into large clumps, there is an increase in light transmittance. Different aggregation-inducing agents stimulate different pathways of activation and different patterns of aggregation are observed. The main drawback of the optical method is that it must be performed on PRP, necessitating the separation of platelets from red blood cells and adjustment of the platelet count to a standardized value.

Impedance detection can be used to test anti-coagulated blood with no need to isolate platelets from other components of the blood, although in many cases the sample is diluted before testing. The method detects aggregation by passing a very small electric current between two electrodes immersed in a sample of blood (or PRP) and measuring electrical impedance between the electrodes. During initial contact with the blood or PRP, the electrodes become coated with a monolayer of platelets. If no aggregating agent is added, no further interactions occur between the platelets and the electrodes and electrical impedance remains constant. When an aggregation inducing agent is added, platelets aggregate on the electrodes and there is an increase in impedance.

The CHRONO LOG Model 530 and Model 540 use the optical method for PRP and the impedance method for whole blood aggregometry. Various photometers are commercially available for measuring the light absorbance of liquid samples in microtitration plates

In one embodiment, epinephrine-induced platelet aggregation is assessed using SPIONs in vitro. In one embodiment, a sample of fresh whole blood is taken in 3.2% trisodium citrate (9 parts of whole blood, 1 part of trisodium citrate), mixed well and incubated at 37° C. for 10 to 15 minutes. Then, the samples are centrifuged at 200 g for 10 minutes to obtain rich plasma (PRP). Then SPIONs are incubated with PRP at 37° C. for 30 min with stirring (1000 rpm) followed by addition of 10 μM epinephrine. The aggregation of the sample is assessed using optical aggregometry.

While not wishing to be limited by theory, embodiments of these methods are based on the observation that the anti-platelet activity of SPIONS is more active for patients that respond well to other anti-platelet drugs. For example, SPIONs exhibit less anti-platelet activity in patients where large scale aggregation is not typically reverse by aspirin. On the other hand, SPIONs have more activity in patients where large scale aggregation is stopped or slowed by aspiring. For aggressive epinephrine induced aggregation or for a drug resistant patient, citrate functionalized SPIONs can slow the aggregation kinetics, but cannot completely block it. Consequently, in vitro assays on samples from a patient with SPIONs can provide an indication whether the patient will respond to therapy with SPIONs and/or aspirin. Information obtained from these assays can also indicate whether the physician can increase the dose of the antiplatelet drug.

The present invention, thus generally described, will be understood more readily by reference to the following examples, which are provided by way of illustration and are not intended to be limiting of the present invention.

### EXAMPLES

### Example 1

### Preparation and Characterization of the SPIONS

In this example, an illustrative method for making functionalized iron oxide nanoparticle is described. Magnetic iron oxide nanoparticles were prepared by co-precipitating 2 g ferrous chloride and 4 g ferric chloride (solubilized in 50 ml 2N HCl) by 150 ml 1.5(N) sodium hydroxide upon constant stirring at room temperature. The precipitate was washed well with milli-Q water and 20 ml citrate buffer (1.6 g citric acid and 0.8 g tri-sodium citrate) was added to collect the stabilized nanoform in solution at a pH around 6.3. All these steps were performed in presence of a strong bar magnet to facilitate the process.

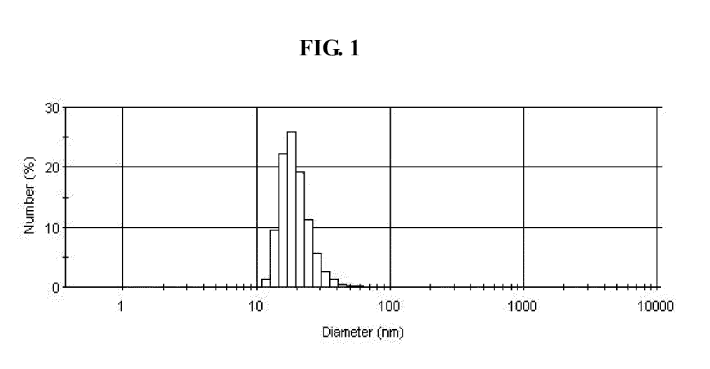

Nanoparticle size was determined by Photon Correlation Spectroscopy (PCS) using the Nano-ZS (Malvern) instrument equipped with a 4 mW He—Ne Laser (λ=632 nm). The magnetic properties were characterized by Superconducting Quantum Interference Device (SQUID) using MPMS-7 (Quantum Design). The particle size distribution by dynamic light scattering of the super paramagnetic iron oxide nanoparticle used for the study is shown in FIG. 1. FIG. 1 shows the plasmon behavior of the citrate-capped nanoparticle. The synthesized nanoparticles (synthesized by the route of synthesis shown above) had low polydispersity ˜0.27 and number distribution ˜20 nm.

Variation of magnetization (M) versus applied magnetic field (H) of the sample was measured at 5K, 100K, 200K and 300K up to 4 T and the measurement was performed in Superconducting Quantum Interference Device (SQUID). The measurements using SQUID are based on the principle of Brownian motion. Particles, emulsions and molecules in suspension undergo Brownian motion. This is the motion induced by the bombardment by solvent molecules that themselves are moving due to their thermal energy. If the particles or molecules are illuminated with a laser, the intensity of the scattered light fluctuates at a rate that is dependent upon the size of the particles as smaller particles are “kicked” further by the solvent molecules and move more rapidly. Analysis of these intensity fluctuations yields the profile of the autocorrelation function. The decay of the autocorrelation function is exponential in nature, with the exponent proportional to the diffusion coefficient of the particle, which in turn in dependent on the particle size.

The calculated value of the diameter (from blocking temperature) of the synthesized nanoparticles was ˜16 nm. The calculated value of the diameter of the iron oxide nanoparticle was determined using the equation TB=K*V/25 kB, where the magnetocrystalline anisotropy constant (K) of iron oxide nanoparticle varies from 1.4−5*10⁵ J/m³, TB is the blocking temperature (80K), and kB is the Boltzman constant. The calculated value of the synthesized nanoparticles was is in good agreement with the experimentally found diameter (˜20 nm) through dynamic light scattering (DLS) study. DLS is sometimes referred to as Photon Correlation Spectroscopy (PCS) or Quasi-Elastic Light Scattering (QELS), is a non-invasive, technique for measuring the size of molecules and particles typically in the submicron region, and with the latest technology lower than 1 nanometre.

The physical characterization of the synthesized iron-oxide nanoparticle was performed from dynamic light scattering measurement and also from low temperature magnetization studies. The synthesized nanoparticles had low polydispersity ˜0.27 and number distribution ˜20 nm as explained above. There was a small population of large size particles, detectable only in the intensity distribution. The low polydispersity value implied pre-dominance of a single population.

### Example 2

### Inhibition of Platelet Aggregation by SPIONs

In this example, platelet aggregation in the presence of SPIONs and various agonists was examined. The study was carried out using chronolog optical aggregometry.

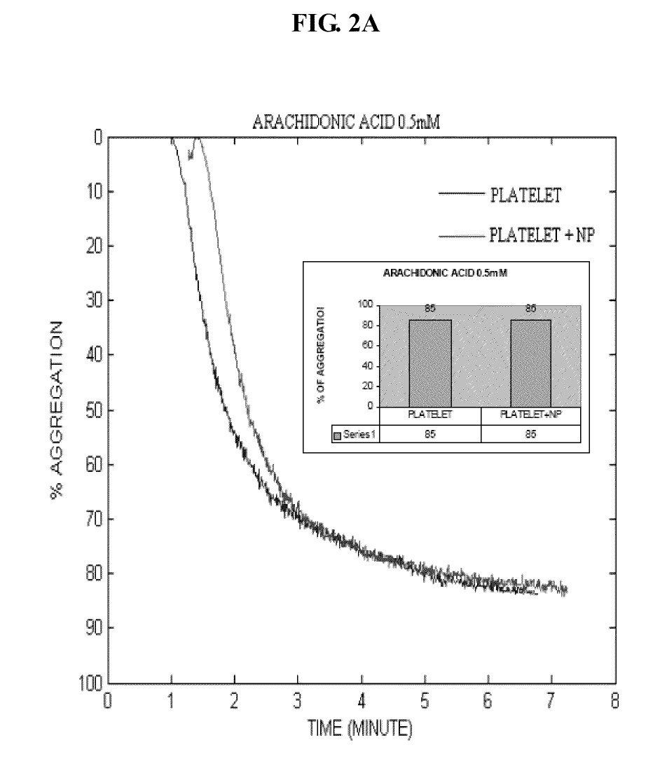

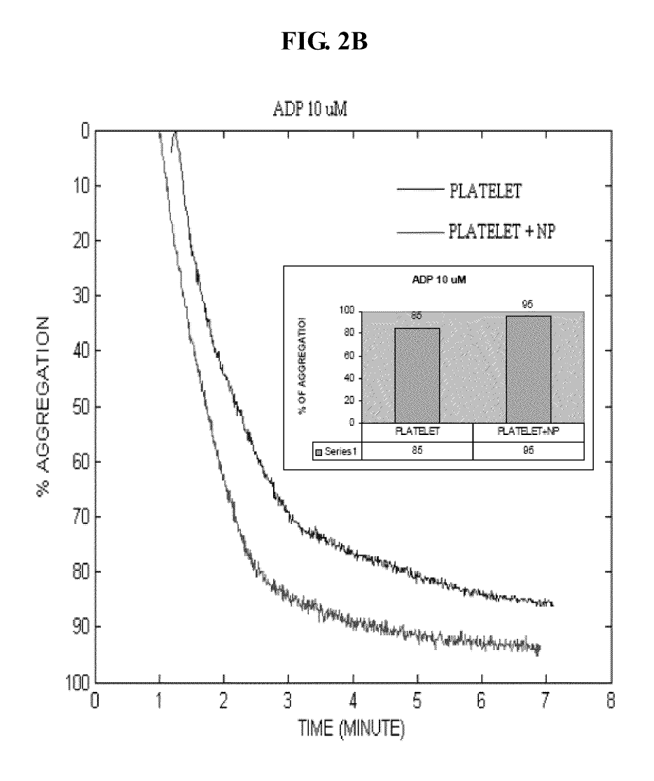

FIGS. 2A and 2B respectively represent aggregometric results in presence of the agonists arachidonic acid (0.5 mM) and ADP (10 μM). Arachidonic acid is the main target for the antiplatelet drug aspirin and ADP is the main target for the antiplatelet drug clopidogrel. FIGS. 2A and 2B show that in the presence of SPIONs, there is no significant change in aggregation in presence of these two platelet agonists. Thus, SPIONs cause no change in ADP- and arachidonic acid-induced aggregation of platelets from normal healthy individuals. The 85% to 95% aggregation increase in FIG. 2B can be ignored as the fractional increase is insignificant.

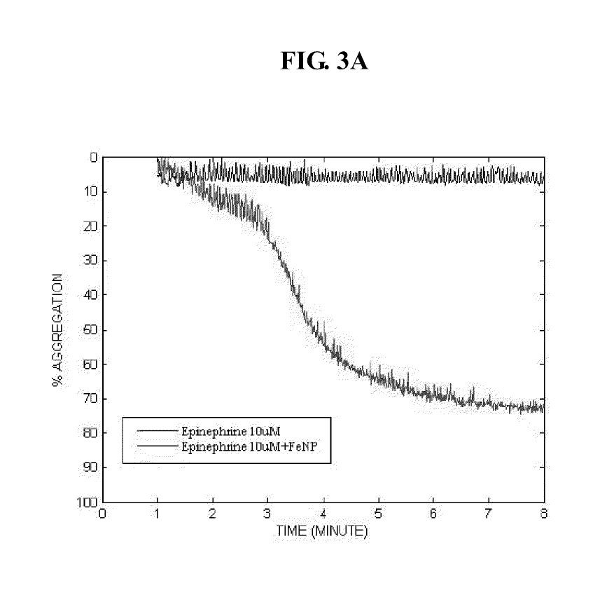

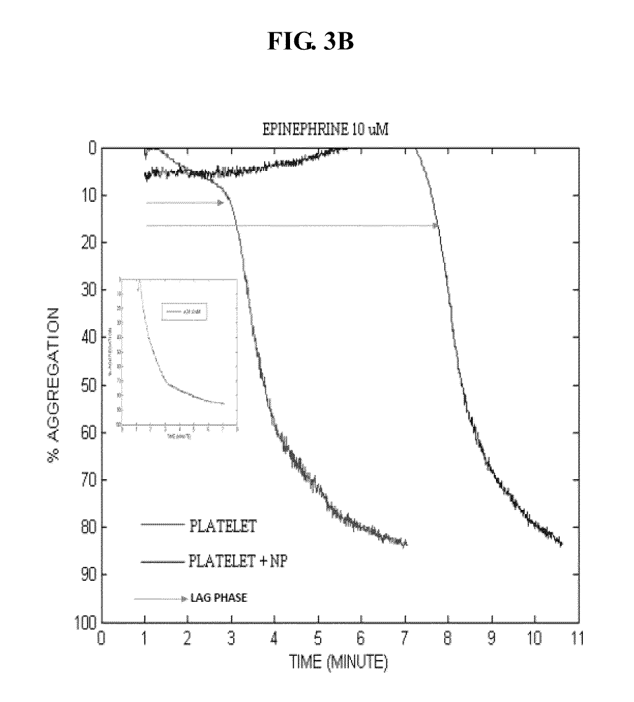

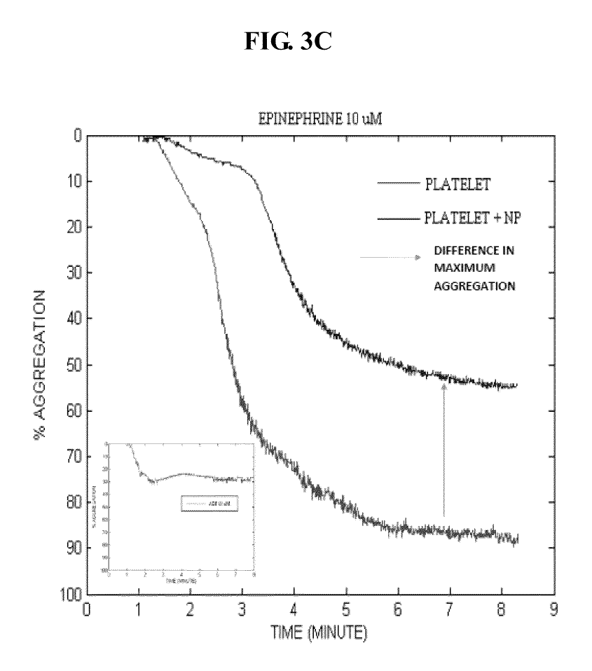

Next, the aggregation of platelets by epinephrine (10 μM final concentration) in the presence and absence of SPIONs was examined. FIG. 3A shows the aggregometric result of epinephrine-induced platelet aggregation in a normal individual. In presence of SPIONs, the aggregation is totally blocked. The conclusion is that the SPIONs are a specific inhibitor for platelet aggregation induced by the agonist epinephrine.

FIG. 3B shows the effect of SPIONs on an ACS patient. A sample of fresh whole blood was taken in 3.2% trisodium citrate (9 parts of whole blood, 1 part of trisodium citrate), mixed well and incubated at 37° C. for 10 to 15 minutes. Then, the samples were centrifuged at 200 g for 10 minutes to obtain rich plasma (PRP). Then SPIONs are incubated with PRP at 37° C. for 30 min with stirring (1000 rpm) followed by addition of 10 μM epinephrine. Platelet aggregation by epinephrine (10 μM final concentration) in the presence and absence of SPIONs, before clopidogrel treatment is shown. The inset figure shows ADP induced aggregation in a patient not treated with clopidogrel. On the sample treated with SPIONs, there is a delay in aggregation. As such, the administration of SPIONs to a subject is useful alone or in combination with other anti-platelet agents to inhibit or delay platelet aggregation.

FIG. 3C shows platelet aggregation by epinephrine (10 μM final concentration) in presence and absence of SPIONs, after clopidogrel treatment. The inset figure shows ADP cannot induce aggregation in a patient treated with clopidogrel. FIG. 3C shows that when the patient was administered clopidogrel, the drug-like effect of the SPION is clearly observed. The delay has disappeared and there is an actual reduction in the activity of the platelets.

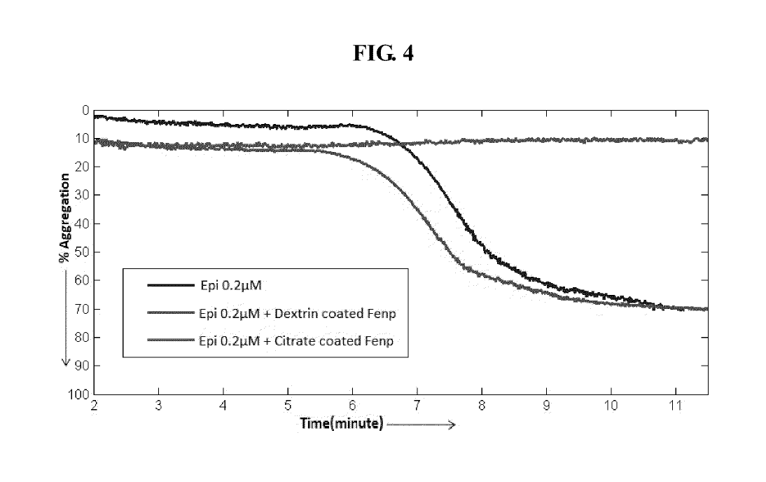

FIG. 4 presents a graph showing the effect of dextrin-functionalized SPIONs on platelet aggregation. is a graph showing the effects of illustrative dextrin functionalized SPIONs on platelet aggregation. Magnetic iron oxide nanoparticles were prepared by co-precipitating 2 g ferrous chloride and 4 g dextrin (solubilized in 50 ml 2N HCl) by 150 ml 1.5(N) sodium hydroxide upon constant stirring at room temperature. The precipitate was washed well with milli-Q water. All these steps were performed in presence of a strong bar magnet to facilitate the process. These results show that the dextrin-functionalized nanoparticle were not as effective in inhibiting platelet aggregation as the citrate-functionalized nanoparticles.

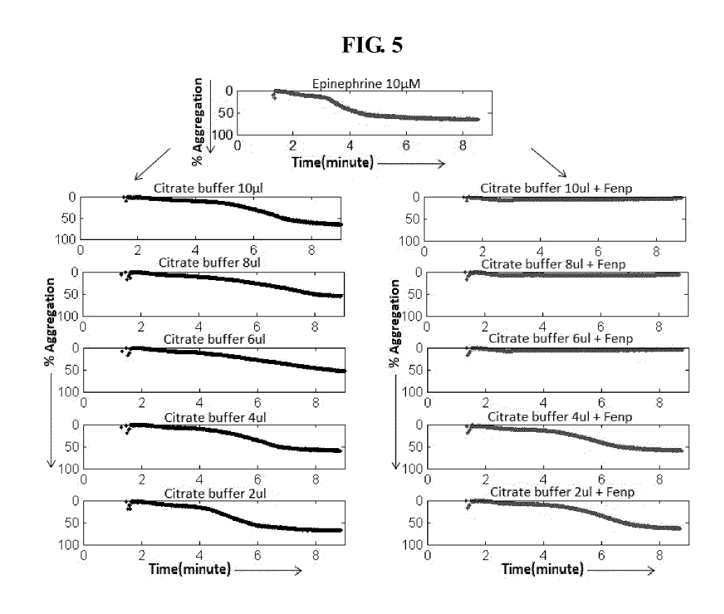

FIG. 5 is a series of graphs showing the effects of illustrative citrate functionalized SPIONs and citrate buffer on platelet aggregation. The results show that the combination of citrate buffer enhances the anti-platelet effects of citrate-functionalized SPIONs.

While certain embodiments have been illustrated and described, it should be understood that changes and modifications can be made therein in accordance with ordinary skill in the art without departing from the technology in its broader aspects as defined in the following claims.

The embodiments, illustratively described herein, may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, include the number recited and refer to ranges which can be subsequently broken down into subranges as discussed above. Finally, as will be understood by one skilled in the art, a range includes each individual member.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for inhibiting epinephrine-induced platelet aggregation in a subject in need thereof, the method comprising: administering to the subject an effective amount of one or more superparamagnetic iron oxide nanoparticles (SPIONs).

2. The method of claim 1, wherein the SPION at least partially inhibits an epinephrine signaling pathway, thereby inhibiting epinephrine-induced platelet aggregation.

3. The method of claim 1, wherein the SPION delays epinephrine-induced platelet aggregation.

4. The method of claim 1, wherein the one or more SPIONs are functionalized SPIONs.

5. The method of claim 4, wherein the functionalized SPIONs are functionalized with citrate.

6. The method of claim 1, wherein the one or more SPIONs have a polydispersity from about 0.25 to about 0.29.

7. The method of claim 1, wherein the one or more SPIONs have a mean diameter from about 18 nm to about 22 nm.

8. The method of claim 1 further comprising administering to the subject one or more additional anti-platelet aggregation agents.

9. The method of claim 8, wherein the one or more additional anti-platelet aggregation agents are selected from the group consisting of: aspirin, clopidogrel, ticlopidine, abciximab, dipyridamole.

10. The method of claim 1 further comprising applying a magnetic field to a body region of the subject in order to enhance the accumulation of the one or more SPIONs in the body region.

11. The method of claim 1, wherein the subject is an acute coronary syndrome patient.

12. The method of claim 1, wherein the subject is suffering from a thrombotic disorder.

13. The method of claim 12, wherein the thrombotic disorder is selected from the group consisting of acute thrombotic stroke, venous thrombosis, myocardial infarction, unstable angina, abrupt closure following angioplasty or stent placement, and thrombosis as a result of peripheral vascular surgery.

14. A method for sensing platelet function, the method comprising:
contacting a first sample of platelets from a subject with an agonist of platelet aggregation;
contacting a second sample of platelets from the subject with the agonist of platelet aggregation and one or more superparamagnetic iron oxide nanoparticles (SPIONs);
measuring the aggregation of the platelets in the first sample and the second sample; and
comparing the aggregation of platelets between the first sample and the second sample to determine the response of the sample of platelets to the agonist.

15. The method of claim 14, wherein the subject comprises a normal subject or an acute coronary syndrome patient.

16. The method of claim 14, wherein the agonist is selected from the group consisting of: epinephrine, ADP and collagen.

17. The method of claim 14, wherein the subject is an acute coronary syndrome patient that has been administered an antiplatelet aggregation agent.

18. The method of claim 17, wherein the anti-platelet aggregation agent is selected from the group consisting of: aspirin, clopidogrel, ticlopidine, abciximab, dipyridamole.

19. The method of claim 14 further comprising comparing the measured response of the sample of platelets to a reference sample in order to detect an aberrant platelet function.

20. (canceled)

21. The method of claim 14, wherein measuring the aggregation of the platelets is by optical aggregometry.
